(19) United States
(12) Patent Application Publication
(10) Pub. No.: US 2026/0061121 A1
(43) Pub. Date: Mar. 5, 2026

(54) ENHANCED PATCH PUMP AND COMBINATION CONTROL SYSTEMS WITH ALARM ANNUNCIATION AND PRIORITIZATION AND BIHORMONAL VERSIONS WITH GLUCAGON

(71) Applicant: Beta Bionics, Inc., Irvine, CA (US)

(72) Inventors: Mike Rosinko, Peoria, AZ (US); Mike Mensinger, San Diego, CA (US); David Lim, Irvine, CA (US)

(73) Assignee: Beta Bionics, Inc., Irvine, CA (US)

(21) Appl. No.: 19/317,389

(22) Filed: Sep. 3, 2025

Related U.S. Application Data
(60) Provisional application No. 63/689,936, filed on Sep. 3, 2024.

(51) Int. Cl. A61M 5/142; A61K 38/26; A61K 47/20

(52) U.S. Cl. CPC A61M 5/14248; A61K 38/26; A61K 47/20; A61M 2005/14252; A61M 2202/0007; A61M 2202/04; A61M 2205/18; A61M 2205/3561; A61M 2205/502; A61M 2205/581; A61M 2205/582; A61M 2205/583

(57) ABSTRACT

The iLet BIONIC PANCREAS system improves alarm annunciation and prioritization with the enumeration of conditional improvements for pairing of, for example, the iLet APP & iLet BIONIC PANCREAS (along with patch pumps in half-of a dozen embodiments) respecting silence conditions for alarms; learning glucagon response; real time mirrored display, data sharing; pairing processes; confidence reminders; widgets; and iLet APP control features for bolusing meal announcements; user providing input and changes mind; and ILet APP therapy Restore.

### CROSS-REFERENCE TO RELATED APPLICATIONS

This document claims the benefit of priority to U.S. Provisional Application Ser. No. 63/689,936, filed Sep. 3, 2024, which is hereby incorporated by reference in its entirety.

### OBJECTS AND SUMMARY OF THE INVENTIONS

According to embodiments, there are disclosed Novel enhanced tubeless insulin pumps, comprised of, in combination, at least a three day wear time (with a 12 hour grace period); at least 200 units of insulin capacity; smart phone controlled; re-usable and charge-free; automatically paired; and equipped with a least a 4.5 mm cannula to trigger insertion via button actuation.

According to embodiments alarms which are triggered or annunciated for less critical functions, benign to insulin flow, cannot impact this critical feature. Multiple schema are provided to coordinate any number of different implementations of the patent applications and patents of the instant assignee.

### BRIEF DESCRIPTION OF THE DRAWINGS

FIG. 1 shows a cartooned schematic of an embodiment of a patch pump functioning as part of the system for at least one of Bihormonal Glucagon Patch, multi-patch type of set-up and component of a third party insulin delivery system;

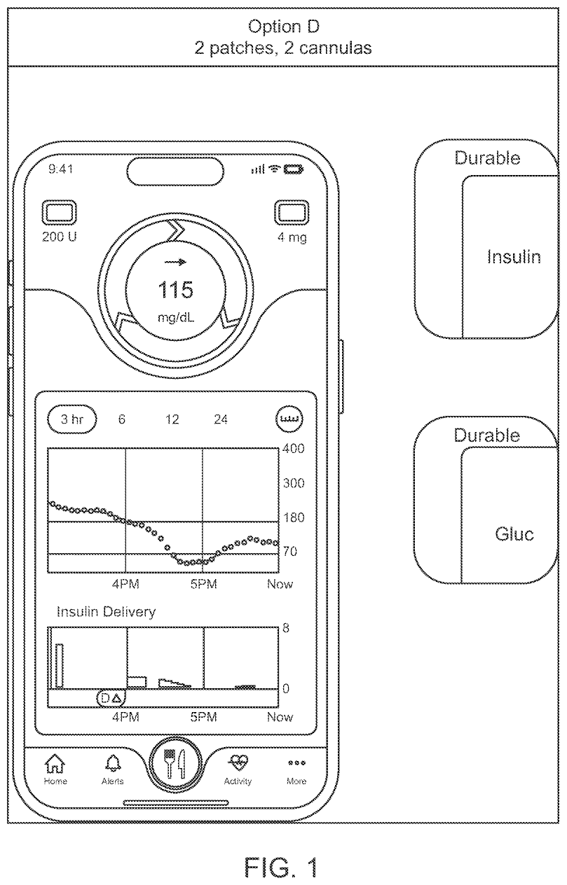

FIG. 2 shows a cartooned schematic of an embodiment of a patch pump functioning as part of the system for at least one of Bihormonal Glucagon Patch, multi-patch type of set-up and component of a third party insulin delivery system;

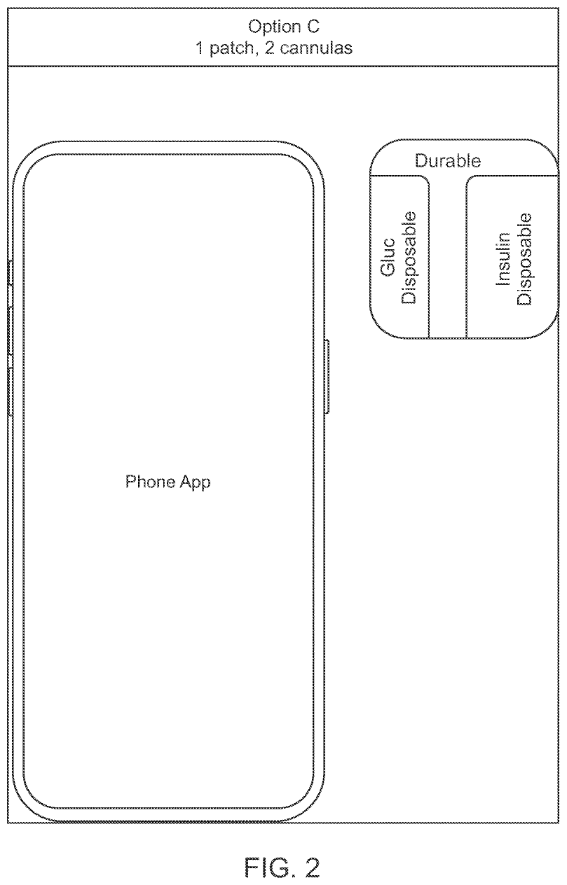

FIG. 3 shows a cartooned schematic of an embodiment of a patch pump functioning as part of the system for at least one of Bihormonal Glucagon Patch, multi-patch type of set-up and component of a third party insulin delivery system;

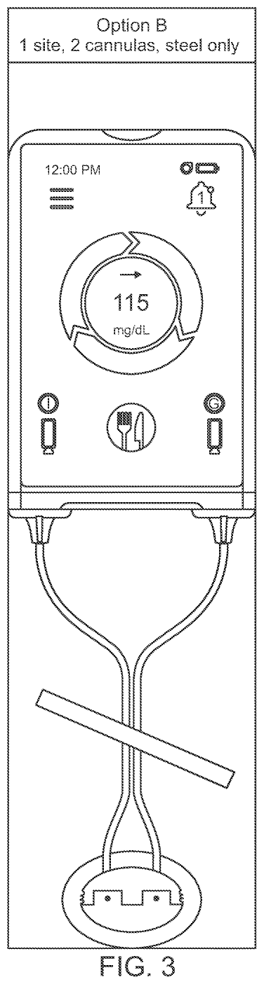

FIG. 4 shows a cartooned schematic of an embodiment of a patch pump functioning as part of the system for at least one of Bihormonal Glucagon Patch, multi-patch type of set-up and component of a third party insulin delivery system;

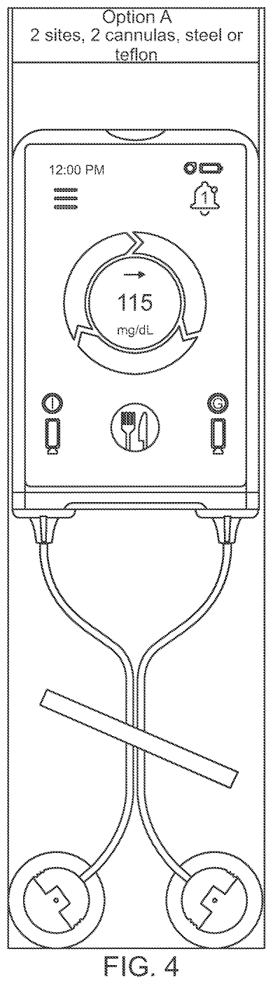

FIG. 5 shows a cartooned schematic of an embodiment of a patch pump functioning as part of the system for at least one of Bihormonal Glucagon Patch, multi-patch type of set-up and component of a third party insulin delivery system;

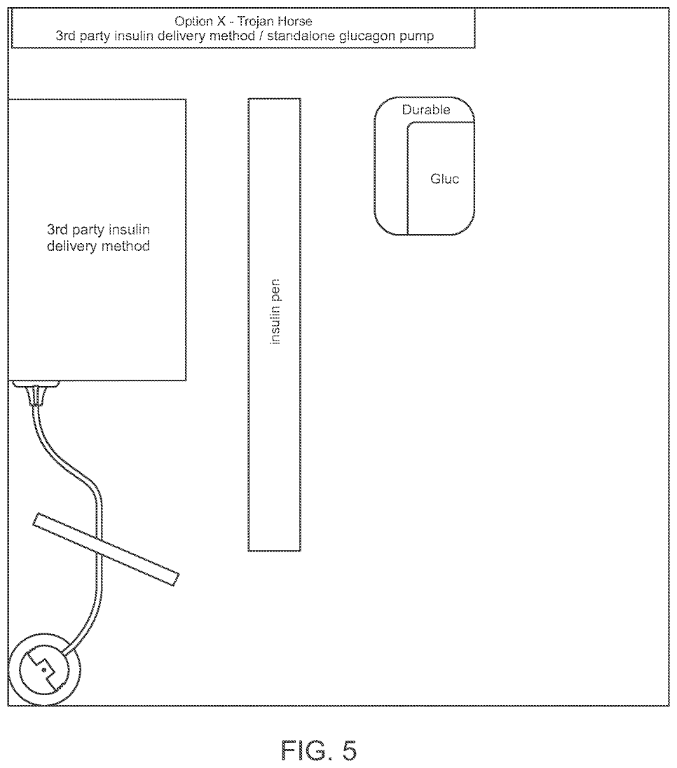

Briefly stated, new methods for adapting patch pumps to human factors are disclosed whereby priming doses with bodyweight are the only thing required. Additionally, in a bihormonal implementation, the meal dose may be further adapted based on the amount of glucagon delivered for example reducing the meal size if glucagon is delivered.

During the management of everything from glucose excursions activities are monitored and the meal bolus associated with this qualitative announcement is increased or decreased if the glucose values or pattern is higher or lower than expected, including improved algorithmic derivations of these data sets and glucose values.

According to embodiments, there are provided patch pump systems and methods whereby a user enters a qualitative meal announcement (for example B, L, D+S, M, L) or a algorithmically detected meal. The system initially gives a meal bolus which is conservative and based on rules of thumb, such as based on patient demographics e.g. body mass or TDD. During the post-prandial period, the glucose excursions are monitored and the meal bolus associated with this qualitative announcement is increased or decreased if the glucose value is higher or lower than expected.

According to embodiments, there are provided patch pump systems which titrate meal doses based at least in part on post-prandial glucose excursions, further comprising an ambulatory medicament device configured to generate a dose control signal for delivery of medicament to a subject, the ambulatory medicament device comprising, a medicament delivery interface configured to operatively connect to a medicament pump for infusing medicament into the subject; a display interface configured to output display signals configured to generate user interface screens on a display device; a memory configured to store specific computer-executable instructions; and a hardware processor in communication with the memory and configured to execute the specific computer-executable instructions to at least: generate the dose control signal using a control algorithm employing control parameters, wherein at least one control parameter of the control parameters is driven by meal adaptations using post-prandial insulin evaluation.

According to embodiments, there are disclosed methods for delivering with patch pumps improved approaches to adapting meals using post-prandial insulin, comprising, in combination, delivering a meal bolus and establishing a post-prandial evaluation period; estimating an expected or an ideal total glucose deviation based on meal size and insulin dose; comparing actual total glucose to expected value; ranking minimum glucose and glucose at end of period; and adjusting meal boluses up or down. Likewise, there are disclosed methods for adapting meals using post-prandial insulin comprising, in combination, delivering a meal bolus and establishing a post-prandial evaluation period; evaluating glucose levels during this period; comparing actual total glucose to expected values; potentially replacing traditional bolus calculations in whole or in part; and adjusting meal boluses up or down.

According to embodiments, there are disclosed durable and disposable patch pump combinations for methods of determining if meal dose or correction dose or basal rate needs to be adjusted, driven by an algorithm according to at least the following steps, if a user doesn't announce a meal, system detects meals automatically and doses for a meal; wherein a user announces a meal generally (no meal type or size); wherein there is a fixed dose—user announces type of meal (e.g. quantified by category such as breakfast, lunch, dinner, snack, and the like); said meal size estimation being at least one of: small, medium, large, extra large, and the like, including Meal Type and Size (for example—Small and Breakfast); Carb entry and/or encompassing values for a Specific food—for example—pizza, ice-cream and the like. In the case of the user does not announce a meal, the system may detect the meal automatically and classify the meal based on time of day or the glucose response of the meal or other system detected characteristics. This class of meals may be adapted using the techniques described herein. This classification may include indication of faster or slower acting carbs in addition to indications of the total number carbs consumed in the meal.

### DETAILED DESCRIPTIONS OF PREFERRED EMBODIMENTS

The present inventions support valuable improvements absent within the prior art. For example, within extent systems, a relatively benign error like a speaker failure issue results in a more serious issue since insulin delivery is halted. Prior to the instant system this was an issue—now it is solved. By way of first, non-limiting example, this means if the TANDEM T-slim pump detects an issue (malfunction) of the speaker, vibe motor etc. the pump issues a malfunction error message and stops all insulin delivery and communication with the CGM. So, a relatively benign error like the speaker issue results in a more serious issue since insulin delivery is halted. Likewise, Minimed and Insulet pumps behave in the same way.

The iLet does not do this. If there is a detected speaker issue, for example, the iLet will alert the user to call customer service to have the pump replaced but will maintain insulin delivery. The user is then reminded every 24 hours (I think they are reminded at 4:00 PM every day). This allows the user to receive a pump replacement without having to change to MDI or an alternative insulin delivery method.

The present inventors have discovered and herein disclose novelties driven by a new paradigm whereby patch pumps embody all of the features embodied in the software and logic structures emplaced within and transmitted to extant hardware developed for adaptive learning, with simplified and generified access to third party systems. By integrating instructions for how to fill and prime a patch pump the instant system makes patent each of the following:

In short, among other things, the present inventors have created a system that supplants the strict need for any of these prior art mechanisms to drive fluid from pumps for delivering medicaments to patients with any and all manners of pumps, used for example, to deliver therapy for diabetes. This extends from conventional systems to patch pumps and later developed technologies. To make this patent, both the evolution of said pumps, and the instant improvements are explained herein, to connect the improvements to the historical developments, it is hereby earnestly solicited that such step changes are patentable as new, novel and non-obvious.

Driving the prior art was the fact that by using a clutch mechanism, the engagement between the leadscrew and the nut occurs at assembly, and thus no rotation is needed for the nut to engage the leadscrew by operation of the device. This reduces the number of fluid path prime pulses to prime the pump and assures a full and proper priming of the fluid path before placement on the body. The clutch mechanism also enabled the changing of thread pitch for other drug applications without a need to redesign the tilt nut used in fluid driving mechanisms in other existing pumps.

With the instant inventions, from priming methods through all types of fluid delivery, with both disposable and durable systems power management is improved enabling both rechargeable non-rechargeable paradigms.

1. Novel enhanced tubeless insulin pumps, comprised of, in combination, at least a three-day wear time (with a 12 hour grace period); at least 200 units of insulin capacity; smart phone controlled; re-usable and charge-free; automatically paired; and equipped with a least a 4.5 mm cannula to trigger insertion via button actuation.

2. An improved system, wherein when an App is paired with a Patch Pump:
the app will be the primary device for alert annunciation, and all alerts will annunciate (sound and vibration) on the app.

3. The improved system of claim 2, further comprising, in combination:
when an app is paired with patch pump iterations (as follows):
a user can choose which device will be primary for alert annunciation (sound and vibration) whereby a default alert annunciation will be on the patch pump.

4. The improved system of claim 3, further comprising, in combination:
when an app is paired with patch pump iterations (as follows):
a user can choose to have the alert annunciate on the secondary device if alerts are not resolved in a certain timeframe on the primary device; If a user does not acknowledge an alert on the primary device, the alert annunciation will occur on the secondary device.

5. The improved system of claim 4, further comprising, in combination:
when an app is paired with patch pump iterations (as follows):
clearing an alert on one device (whether through acknowledgement or resolution of the underlying issue) will clear the alert on the other device.

6. The improved system of claim 5, further comprising, in combination:
when an app is paired with patch pump iterations (as follows):
all alerts will always be displayed on the UI of both devices regardless of annunciation settings.

7. The improved system of claim 6, further comprising, in combination:
when an App is paired with patch pump iterations (as follows):
if the patch pump and app are disconnected, all alerts shall revert to annunciating on the patch pump at the user set volume level.

8. The improved system of claim 7, whereby a relatively benign error like a speaker failure issue cannot result in a more serious issue whereby insulin delivery is halted, and fail-safe indications to prevent the same.

9. A patch pump or other ambulatory medical device Bihormonal system being further comprised of:
novel enhanced glucagon-related formulations using at least dimethyl sulfoxide (DMSO) and similar solvents to suspend glucagon in a non-aqueous matrix, for use as the secondary active to insulin for actuation of blood sugar controls.

10. The patch pump or other ambulatory medical device of claim 9, being further comprised of:
Novel enhanced glucagon-related formulations using at least dimethyl sulfoxide (DMSO) and similar solvents to suspend glucagon in a non-aqueous matrix;
whereby said formulation prevents hydrolytic and conformational degradation, resulting in a stable solution.

11. The patch pump or ambulatory medical device of claim 9, being further comprised of:
Novel enhanced glucagon-related formulations using at least dimethyl sulfoxide (DMSO) and similar solvents to suspend glucagon in a non-aqueous matrix; and at least said formulation can be packaged into auto-injectors or pumps and remains stable at room temperature for extended periods, potentially years, along with compliant interfacing and active support foundations.

12. Bihormonal glucagon patch control governed in, at least one of the following configurations:
2 patches, 2 cannulas;
1 patch, 2 cannulas;
1 site, 2 cannulas, steel only;
2 sites, 2 cannulas steel or Teflon.

13. Bihormonal glucagon patch control of claim 12 governed in, at least one of the following configurations:
standalone glucagon pump & 3^{rd} party insulin delivery;
2 patches, 2 cannulas;
1 patch, 2 cannulas;
1 site, 2 cannulas, steel only;
2 sites, 2 cannulas steel or Teflon.

14. Novel enhanced alarm annunciation and prioritization being AUTO-MAGICAL or it just works when a user walks away from app it switches automatically to the pump.

15. Novel enhanced alarm annunciation and prioritization according to claim 14, further comprising the condition state of disconnection all alerts being reversion to user set volume level.

16. Novel enhanced alarm annunciation and prioritization of claim 15, being identical to the current ILET BIONIC PANCREAS SYSTEM.

17. Novel enhanced alarm annunciation and prioritization of claim 16, wherein if iLET device does not receive annunciation from the phone then the pump would need to annunciate.

18. Novel enhanced alarm annunciation and prioritization of claim 17, comprised of revisions with two options, user selected:
some alerts where it clears on both displays—others where it does not.

19. Novel enhanced alarm annunciation and prioritization of claim 18, further comprised of: Geolocation fencing/triggering.

20. Novel enhanced alarm annunciation and prioritization of claim 19, for LOW INSULIN ALERT; Third party service monitoring and proximity network of IM IN NEED comprising raising a flag in at least one the Bionic Circle and Bionic Universe, or analogous third party systems.
